SEQUENCE LISTING (26 sequences)

SEQ ID NO: 1 - protein, 4 aa, Homo sapiens
  MISC_FEATURE (4)..(4): Amidated Phenylalanine
YPWF

SEQ ID NO: 2 - protein, 4 aa, Homo sapiens
  MISC_FEATURE (4)..(4): Amidated Phenylalanine
YPFF

SEQ ID NO: 3 - protein, 4 aa, Artificial Sequence
  Synthetic mu-opiate receptor peptides
  MISC_FEATURE (4)..(4): Hydroxylated Phenylalanine
YPWF

SEQ ID NO: 4 - protein, 4 aa, Artificial Sequence
  Synthetic mu-opiate receptor peptides
  MISC_FEATURE (4)..(4): Hydroxylated Phenylalanine
YPFF

SEQ ID NO: 5 - protein, 4 aa, Artificial Sequence
  Synthetic mu-opiate receptor peptides
  MISC_FEATURE (4)..(4): Amidated Phenylalanine
YPWF

SEQ ID NO: 6 - protein, 4 aa, Artificial Sequence
  Synthetic mu-opiate receptor peptides
YPFF

SEQ ID NO: 7 - protein, 4 aa, Artificial Sequence
  Synthetic mu-opiate receptor peptides
  MISC_FEATURE (3)..(3): Nitrosylated Tryptophan
  MISC_FEATURE (4)..(4): Amidated Phenylalanine
YPWF

SEQ ID NO: 8 - protein, 4 aa, Artificial Sequence
  Synthetic mu-opiate receptor peptides
  MISC_FEATURE (3)..(3): Nitrosylated Phenylalanine
  MISC_FEATURE (4)..(4): Amidated Phenylalanine
YPFF

SEQ ID NO: 9 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 10 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 11 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 12 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 13 - protein, 4 aa, Artificial Sequence
  Synthetic mu-opiate receptor peptides
  MISC_FEATURE (2)..(4): Cyclic Peptide
YKWF

SEQ ID NO: 14 - protein, 4 aa, Artificial Sequence
  Synthetic mu-opiate receptor peptides
  MISC_FEATURE (2)..(4): Cyclic Peptide
YKFF

SEQ ID NO: 15 - protein, 4 aa, Artificial Sequence
  Synthetic mu-opiate receptor peptides
  MISC_FEATURE (2)..(4): Cyclic Peptide
  MISC_FEATURE (2)..(2): Ornathine
YXWF

SEQ ID NO: 16 - protein, 4 aa, Artificial Sequence
  Synthetic mu-opiate receptor peptides
  MISC_FEATURE (2)..(4): Cyclic Peptide
  MISC_FEATURE (2)..(2): Ornathine
YXFF

SEQ ID NO: 17 - protein, 4 aa, Artificial Sequence
  Synthetic mu-opiate receptor peptides
  MISC_FEATURE (2)..(4): Cyclic Peptide
  MISC_FEATURE (3)..(3): Nitrosylated Tryptophan
YKWF

SEQ ID NO: 18 - protein, 4 aa, Artificial Sequence
  Synthetic mu-opiate receptor peptides
  MISC_FEATURE (2)..(4): Cyclic Peptide
  MISC_FEATURE (3)..(3): Nitrosylated Phenylalanine
YKFF

SEQ ID NO: 19 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Nitrosylated Tryptophan
YXWF

SEQ ID NO: 20 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Nitrosylated Phenylalanine
YXFF

SEQ ID NO: 21 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

SEQ ID NO: 22 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Alkylated Phenylalanine
YXFF

SEQ ID NO: 23 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

SEQ ID NO: 24 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Alkylated Phenylalanine
YXFF

SEQ ID NO: 25 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

SEQ ID NO: 26 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

(19) United States
(12) Patent Application Publication
(10) Pub. No.: US 2020/0331962 A1
(43) Pub. Date: Oct. 22, 2020

(54) NOVEL THERAPEUTIC USES OF MU-OPIATE RECEPTOR PEPTIDES

(71) Applicant: Cytogel Pharma, LLC, Darien, CT (US)

(72) Inventor: Theodore E. MAIONE, Green Island, NY (US)

(21) Appl. No.: 16/921,223

(22) Filed: Jul. 6, 2020

Related U.S. Application Data
(63) Continuation of application No. 16/007,671, filed on Jun. 13, 2018, now abandoned.
Application No. 16/007,671 is a continuation of application No. 14/401,731, filed on Nov. 17, 2014, now abandoned.
(60) Provisional application No. 61/649,026, filed on May 18, 2012.

(51) Int. Cl. C07K 5/107; A61K 38/07

(52) U.S. Cl. CPC C07K 5/1016; A61K 38/07

(57) ABSTRACT

The present invention pertains to novel uses of endomorphine-1 peptide, analogs, and salts thereof for therapy of children, patients currently suffering from drug addiction, patients prone to opioid addiction, and patients with chemo-induced pain.

### CROSS REFERENCE TO RELATED APPLICATIONS

This application is a continuation of co-pending U.S. application Ser. No. 16/007,671, filed Jun. 13, 2018; which is a continuation of U.S. application Ser. No. 14/401,731, filed Nov. 17, 2014; which is a National Stage Application of International Application No. PCT/US2013/041617, filed May 17, 2013; which claims priority to U.S. Provisional Application Ser. No. 61/649,026, filed May 18, 2012; all of which are incorporated herein by reference in their entirety.

The Sequence Listing for this application is labeled “As-filed_ST25.txt”, which was created on May 17, 2013, and is 8 KB. The entire contents of this sequence listing is incorporated herein by reference in its entirety

### BACKGROUND OF INVENTION

The major putative function for opiates is their role in alleviating pain. Opioid analgesics provide symptomatic treatment of pain arising from a wide variety of disease states, and are usually required for controlling severe pain, such as pain associated with trauma, myocardial infarction, surgery, and cancer. Other areas where opioid analgesics are well-suited for use in treatment are conditions relating to gastrointestinal disorders, schizophrenia, obesity, high blood pressure, convulsions, coronary artery disease, and seizures.

Unfortunately, use of opioids is associated with serious adverse effects, including sedation, euphoria, respiratory depression, and peripheral vasodilatation. Opioid use also leads to opioid tolerance, dependence, and potential addiction, misuse or diversion. Tolerance is a need for increasing doses to maintain the same pharmacologic effect (in the absence of disease progression or other external factors). Although progressively higher opioid doses may improve pain relief, repeated dose escalation, in many circumstances, has limited utility due to increased side-effects, the lack of incremental benefits with higher doses, the risk of accidental over-dose, and other factors. Opioid tolerance is usually accompanied by a similar degree of physical dependence—a condition in which continued opioid use is required to prevent physical discomfort or withdrawal symptoms including restlessness, lacrimation, rhinorrhea, chills, myalgia, and mydriasis.

Opioid therapy is a high-risk practice and the decision to use opioids, like all medical decisions, is based on a balance of risks and potential benefits. It is generally regarded that opioid therapy should not be given to patients when the potential harms are likely to outweigh therapeutic benefits. According to the Clinical Guidelines for the Use of Chronic Opioid Therapy in Chronic Noncancer Pain recommended by the American Pain Society and the American Academy of Pain Medicine, chronic opioid therapy (COT) should be tapered or discontinued in patients who engage in repeated aberrant drug-related behaviors or drug abuse/diversion. Patients with a personal or family history of drug abuse or alcoholism, psychiatric conditions, or cognitive impairment have a significantly higher likelihood of engaging in aberrant drug-related behaviors after initiation of COT.

Pediatric use of opioid therapy must also be avoided. Use of opioids in younger patients has been associated with higher risks of engaging in aberrant drug-related behaviors. Also, opioid therapy could produce long-term opioid tolerance in infants who are at the critical stage of brain development.

Accordingly, there is a need to provide opioid therapy for high-risk patients, such as children, former addicted patients, and addiction-prone patients. Despite the immediate pain-relief effects and other benefits, it is currently believed that, for these high-risk patients, the potential harms of opioid therapy outweigh its therapeutic benefits.

### BRIEF SUMMARY

The present invention provides novel uses of endomorphine-1 peptide, analogs, and salts thereof for therapy of children, pregnant women, patients prone to opioid addiction, and patients with chemotherapy-induced, or other chronic, pain. Advantageously, it is now discovered that the endomorphin-1 peptide, analogs, and salts of the present invention are highly effective, potent, and rapid-acting on the activation of the mu-opioid receptor, but that they are associated with relatively low tolerance, less severe side-effects, and lower risk of developing physical dependence.

In one embodiment, the methods of the present invention comprise administering, to a subject in need of such treatment, an effective amount of an isolated peptide or salt thereof, wherein the peptide has a general formula: Tyr-X₁-X₂-X₃,

wherein X₁ is Pro, D-Lys or D-Orn,

X₂ is Trp, Phe or N-alkyl-Phe, wherein alkyl has 1 to about 6 carbon atoms, and

X₃ is Phe, Phe-NH₂, D-Phe, D-Phe-NH₂ or p-Y-Phe, wherein Y is NO₂, F, Cl or Br;

wherein the subject is a child, is prone to opioid addiction, and/or has chemotherapy-induced pain.

In a further embodiment, the present invention comprises, prior to administration of the peptide and/or peptide salt of the present invention, the step of determining whether a subject is a child, is prone to opioid addiction, and/or has chronic pain, wherein the peptide and/or peptide salt is administered if the subject is a child, is prone to opioid addiction, and/or has chronic pain.

In certain embodiments, the present invention provides analgesia, relief from gastrointestinal disorders such as diarrhea, neuroinflammation, neurogenic inflammation, and therapy for drug dependence in patients, such as mammals, including humans. In one embodiment, the present invention provides treatment, for a period of at least 6 weeks, to patients with a non-terminal disease.

The present invention also provides pharmaceutical compositions, containing as an active ingredient an effective amount, of one or more peptides according to the formula set forth above, and a non-toxic, pharmaceutically-acceptable carrier or diluent.

### BRIEF DESCRIPTION OF FIGURES

FIG. 1 shows agonist dose-response curve of CYT-1010, morphine, and met-enkephalin on beta-arrestin recruitment upon activation of the mu-opioid receptor.

FIG. 2 shows agonist dose-sponse curve of CYT-1010, morphine, and met-enkephalin on inhibition of cAMP production upon activation of the mu-opioid receptor.

### BRIEF DESCRIPTION OF SEQUENCES

SEQ ID NO:1 is a peptide useful according to the present invention.

SEQ ID NO:2 is a peptide useful according to the present invention.

SEQ ID NO:3 is a peptide useful according to the present invention.

SEQ ID NO:4 is a peptide useful according to the present invention.

SEQ ID NO:5 is a peptide useful according to the present invention.

SEQ ID NO:6 is a peptide useful according to the present invention.

SEQ ID NO:7 is a peptide useful according to the present invention.

SEQ ID NO:8 is a peptide useful according to the present invention.

SEQ ID NO:9 is a peptide useful according to the present invention.

SEQ ID NO:10 is a peptide useful according to the present invention.

SEQ ID NO:11 is a peptide useful according to the present invention.

SEQ ID NO:12 is a peptide useful according to the present invention.

SEQ ID NO:13 is a peptide useful according to the present invention.

SEQ ID NO:14 is a peptide useful according to the present invention.

SEQ ID NO:15 is a peptide useful according to the present invention.

SEQ ID NO:16 is a peptide useful according to the present invention.

SEQ ID NO:17 is a peptide useful according to the present invention.

SEQ ID NO:18 is a peptide useful according to the present invention.

SEQ ID NO:19 is a peptide useful according to the present invention.

SEQ ID NO:20 is a peptide useful according to the present invention.

SEQ ID NO:21 is a peptide useful according to the present invention.

SEQ ID NO:22 is a peptide useful according to the present invention.

SEQ ID NO:23 is a peptide useful according to the present invention.

SEQ ID NO:24 is a peptide useful according to the present invention.

SEQ ID NO:25 is a peptide useful according to the present invention.

SEQ ID NO:26 is a peptide useful according to the present invention.

### DETAILED DISCLOSURE

Advantageously, the endomorphin-1 peptide, analogs, and salts of the present invention are highly effective, potent, and rapid-acting on the activation of the mu-opioid receptor, yet these compounds have a lower capacity to elicit tolerance, they also have less severe side-effects and a lower risk of causing the patient to develop physical dependence. These characteristics make the endomorphin-1 peptide, analogs, and salts of the present invention particularly suitable for therapy of children, patients prone to opioid addiction, and patients with chemotherapy-induced or other chronic pain.

### Peptides

In preferred embodiments, the present invention pertains to the use of peptides that have the general formula: Tyr-X₁-X₂-X₃, wherein X₁ is Pro, D-Lys or D-Orn; X₂ is Trp, Phe or N-alkyl-Phe, wherein alkyl has 1 to about 6 carbon atoms; and X₃ is Phe, Phe-NH₂, D-Phe, D-Phe-NH₂ or p-Y-Phe, wherein Y is NO₂, F, Cl or Br. Some preferred peptides of the invention are:

(SEQ ID NO: 1)
H-Tyr-Pro-Trp-Phe-NH₂
(SEQ ID NO: 2)
H-Tyr-Pro-Phe-Phe-NH₂
(SEQ ID NO: 3)
H-Tyr-Pro-Trp-Phe-OH
(SEQ ID NO: 4)
H-Tyr-Pro-Phe-Phe-OH
(SEQ ID NO: 5)
H-Tyr-Pro-Trp-D-Phe-NH₂
(SEQ ID NO: 6)
H-Tyr-Pro-Phe-D-Phe-NH₂
(SEQ ID NO: 7)
H-Tyr-Pro-Trp-pNO₂-Phe-NH₂
(SEQ ID NO: 8)
H-Tyr-Pro-Phe-pNO₂-Phe-NH₂
(SEQ ID NO: 9)
H-Tyr-Pro-N-Me-Phe-Phe-NH₂
(SEQ ID NO: 10)
H-Tyr-Pro-N-Et-Phe-Phe-NH₂
(SEQ ID NO: 11)
H-Tyr-Pro-N-Me-Phe-D-Phe-NH₂
(SEQ ID NO: 12)
H-Tyr-Pro-N-Et-Phe-D-Phe-NH₂
(SEQ ID NO: 13)
H-Tyr-c-[D-Lys-Trp-Phe]
(SEQ ID NO: 14)
H-Tyr-c-[D-Lys-Phe-Phe]
(SEQ ID NO: 15)
H-Tyr-c-[D-Orn-Trp-Phe]
(SEQ ID NO: 16)
H-Tyr-c-[D-Orn-Phe-Phe]
(SEQ ID NO: 17)
H-Tyr-c-[D-Lys-Trp-pNO₂-Phe]
(SEQ ID NO: 18)
H-Tyr-c-[D-Lys-Phe-pNO₂-Phe]
(SEQ ID NO: 19)
H-Tyr-c-[D-Orn-Trp-pNO₂-Phe]
(SEQ ID NO: 20)
H-Tyr-c-[D-Orn-Phe-pNO₂-Phe]
(SEQ ID NO: 21)
H-Tyr-c-[D-Lys-N-Me-Phe-Phe]
(SEQ ID NO: 22)
H-Tyr-c-[D-Orn-N-Me-Phe-Phe]
(SEQ ID NO: 23)
H-Tyr-c-[D-Lys-N-Et-Phe-Phe]
(SEQ ID NO: 24)
H-Tyr-c-[D-Orn-N-Et-Phe-Phe]
(SEQ ID NO: 25)
H-Tyr-c-[D-Lys-N-Me-Phe-D-Phe]
(SEQ ID NO: 26)
H-Tyr-c-[D-Lys-N-Et-Phe-D-Phe].

The last fourteen peptides listed are cyclic peptides whose linear primary amino acid sequences are given in SEQ ID NO:13 through SEQ ID NO:26. In this context, the applicants incorporate herein by reference, in its entirety, U.S. Pat. No. 6,303,578.

The peptide of SEQ ID NO:1 is highly selective and very potent for the mu-opiate receptor, with over 4,000-fold weaker binding to delta receptors and over 15,000-fold weaker binding to kappa receptors, thereby reducing the chances of side-effects.

The peptides of this invention may be prepared by conventional solution-phase (Bodansky, M., Peptide Chemistry: A Practical Textbook, 2^{nd} Edition, Springer-Verlag, New York (1993)) or solid phase (Stewart, J. M.; Young, J. D. Solid Phase Peptide Synthesis, 2^{nd} edition, Pierce Chemical Company, 1984) methods with the use of proper protecting groups and coupling agents. A suitable deprotection method may then be employed to remove specified or all of the protecting groups, including splitting off the resin if solid phase synthesis is applied.

Cyclization of the linear peptides can be performed by, for example, substitution of an appropriate diamino carboxylic acid for Pro in position 2 in the peptides through ring closure of the 2-position side chain amino and the C-terminal carboxylic functional groups. The cyclization reactions can be performed with the diphenylphosphoryl azide method (Schmidt, R., Neuhert, K., Int. J. Pept. Protein Res. 37:502-507, 1991).

Peptides synthesized with solid phase synthesis can be split off the resin with liquid hydrogen fluoride (HF) in the presence of the proper antioxidant and scavenger.

The desired products may be isolated from the reaction mixture by crystallization, electrophoresis, extraction, chromatography, or other means. However, a preferred method of isolation is HPLC. All of the crude peptides can be purified with preparative HPLC, and the purity of the peptides may be checked with analytical HPLC. Purities greater than 95% of the synthesized compounds using HPLC have been obtained.

In a preferred embodiment specifically exemplified herein, the present invention pertains to the therapeutic use of the peptide of SEQ ID NO:13, which has the following structure:

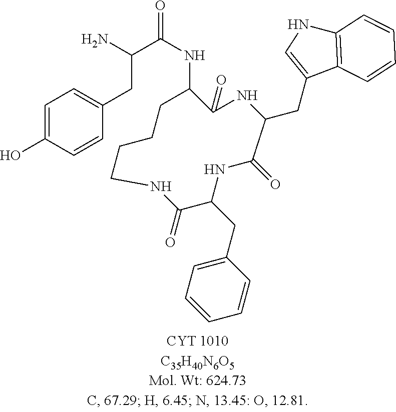 [SMILES: NC(Cc1ccc(O)cc1)C(=O)NC1CCCCNC(=O)C(Cc2ccccc2)NC(=O)C(Cc2c[nH]c3ccccc23)NC1=O]

In a further embodiment, the present invention provides use of salts of the peptides of SEQ ID NOs: 1-26 as active pharmaceutical ingredients. In certain preferred embodiments, acids suitable for preparing the peptide salts are shown in Table 1, and the corresponding peptide salts are shown in Table 2. Preferred salt peptides include maleate salt, hydrochloride salt, lactate salt, aspartate salt, acetate salt, and trifluoro acetate salt.

TABLE 1
Acids
Acetic
Aspartic (L)
Citric
Fumaric
Gluconic (D)
Hippuric
Hydrochloric
Lactic
Malic
Maleic
Mucic
Phosphoric
Sulfuric
Succinic
Tartaric (L)
Trifluoroacetic acid (TFA)

TABLE 2
Salt Forms

| | | | |
|---|---|---|---|
| Acetate | Hippurate | Mucate | Tartrate (L) |
| Aspartate (L) | Hydrochloride | Phosphate | Gluconate (D) |
| Citrate | Lactate | Sulfate | Maleate |
| Fumarate | Malate (L) | Hemi-sulfate | Succinate |
| Trifluoroacetate | | | |

### Therapeutic Use in High-Risk Patients

In one embodiment, the present invention provides opioid therapy to high-risk patients, by administering a pharmaceutical composition comprising an effective amount of one or more peptides of the present invention. The peptides of the present invention can be in a form of the free base, or a salt thereof. In certain embodiments, the present invention is useful for therapy of children, patients prone to opioid addiction, and/or patients with chronic pain. In one embodiment, the present invention provides treatment, for a period of at least 6 weeks, to a patient with a non-terminal disease.

In a further embodiment, the method of the present invention comprises, prior to administration of the peptide and/or peptide salt of the present invention, the step of determining whether a subject is a child, is prone to opioid addiction, is pregnant and/or has chemotherapy-induced, or other chronic, pain, wherein the peptide and/or peptide salt is administered if the subject is a child, is prone to opioid addiction, and/or has chemotherapy-induced pain.

The term “subject” or “patient,” as used herein, describes an organism, including mammals such as primates, to which treatment with the compositions according to the present invention can be administered. Mammalian species that can benefit from the disclosed methods of treatment include, but are not limited to, apes, chimpanzees, orangutans, humans, monkeys; and other animals such as dogs, cats, horses, cattle, pigs, sheep, goats, chickens, mice, rats, guinea pigs, and hamsters. In a preferred embodiment, the subject or patient is a human being.

The term “child,” as used herein, refers to a human subject of less than 18 years of age, and includes an infant. In certain embodiments, the child is less than 14 years old or of any age less than 14 years old, such as less than 12 years old, less than 8 years old, less than 6 years old, less than 5 years old, less than 3 years old, less than 2 years old, or less than 1 year old. The term “infant,” as used herein, refers to a human subject of less than 12 months old. In certain embodiments, the infant is less than 9 months old or of any age less than 9 months old, such as less than 6 months old, less than 3 months old, or less than 1 month old.

The term “drug addiction,” as used herein, refers to its ordinary meaning that is a pattern of drug abuse in which an individual is preoccupied with drug procurement and use, and, thus, for example, neglects other responsibilities and personal relationship, and is usually associated with a high level of drug dependence.

The term “drug abuse,” as used herein, refers to its ordinary meaning that is the use of a drug in a manner that is detrimental to the health or well-being of the drug user, other individuals, or the society as a whole. In certain embodiments, drug abuse includes non-medical use of prescription drug, drug diversion, and illicit use of drugs.

The term “drug dependence,” as used herein, refers to its ordinary meaning that is a condition in which an individual feels compelled to repeatedly administer a drug, such as a psychoactive drug. The condition is caused by positive reinforcement (psychological dependence) and negative reinforcement (physical dependence) from conditioned drug use.

“Psychological dependence” of a drug is believed to be mediated by common neuronal pathway(s) that evoke behavioral reinforcement of drug use. Currently, it is believed that the psychological dependence of different psychoactive drugs (e.g., CNS depressants such as opioids, barbiturates, benzodiazepines, and alcohol; CNS stimulants such as cocaine, nicotine, caffeine) is remarkably similar, despite the varied behavioral and physiological effect that these drugs produce.

Physical dependence, also referred to as neuroadaptation, refers to its ordinary meaning that is a condition in which continued drug use is required to prevent physical discomfort or withdrawal symptoms. Physical dependence results from the adaptation of specific neurons or areas of the brain to the continued presence of a drug.

In one embodiment, patients prone to opioid addiction can be treated in accordance with the present invention. Based on current clinical evidence, patients with a personal or family history of alcoholism or addiction and/or abuse of drugs, more specifically, psychoactive drugs, patients with psychiatric conditions, and patients with cognitive impairment are prone to opioid addiction upon initiation of opioid therapy.

Psychoactive drugs include, but are not limited to, opioid agonists including, but not limited to, morphine, heroin, fentanyl and its derivatives, meperidine, methadone, oxycodone, codeine, hydrocodone, propoxyphene, tramadol, endomorphine, leu-enkephalin, and met-enkephaline; barbiturates and benzodiazepines including, but not limited to, pentobarbital, gamma hydroxybutyrate (GHB); amphetamine and its derivatives including, but not limited to, methamphetamine, methylphenidate, phentermine, and fenfluramine; cocaine; nicotine; caffeine; cannabis and its derivatives; and hallucinogens including, but not limited to, lysergic acid diethylamide (LSD), mescaline, psilocybin, and phencyclidine.

In one specific embodiment, the present invention is provided to a patient with a personal or family history of addiction or abuse of opioids.

In addition, patients prone to opioid addiction may also be diagnosed by genetic testing. It has been recognized that genetic polymorphisms could play a role in an individual's susceptibility to opioid addiction. For example, U.S. Pat. No. 6,337,207 discloses that a variant allele C17T of the human mu opioid receptor gene is present at a statistically significant greater frequency in the genome of at least one defined subset of addicts suffering from at least one addictive disease (e.g., addiction to heroin, alchohol), than in the genomes of people not suffering from such a disease.

In one embodiment, patients prone to opioid addiction include patients with past physical and/or psychological dependence on psychoactive drugs. In certain embodiments, patients prone to opioid addiction have past physical dependence on one or more opioid agonists including, but not limited to, morphine, heroin, fentanyl and its derivatives, meperidine, methadone, oxycodone, codeine, hydrocodone, propoxyphene, tramadol, endomorphine, leu-enkephalin, and met-enkephaline.

In another embodiment, a patient with chronic pain, including, for example, chemotherapy-induced pain, is treated in accordance with the present invention.

In a further embodiment, the present invention comprises, during a period preceding the use of the peptide(s) and/or peptide salt(s) of the present invention, the steps of periodically assessing the therapeutic benefits and adverse effects of a previous therapy with an opiate, wherein the peptide(s) and/or peptide salt(s) is administered if under the previous treatment:

there lacks incremental benefits with higher doses,

the subject develops intolerable side-effects,

the subject is addicted to the opiate, and/or

the subject is engaging in abusive uses of the opiate.

Drug addiction and abuse can be detected by standard clinical protocols and techniques, such as urine drug testing (UDT), prescription monitoring, and random pill counts.

In one embodiment, the peptides and peptide salts of the present invention can be used to treat diseases or conditions in which activation of the mu-opioid receptor is beneficial. In certain embodiments, the present invention provides analgesia.

In certain embodiments, the present invention provides long-term treatment of chronic pain. In certain embodiments, the patients treated in accordance with the present invention suffer from pain lasting at least 1 month after the initial episode, or any time period longer such as at least 6 weeks, 3 months, 6 months, 1 year, 2 years, 3 years, 5 years, 8 years, 10 years, 15 years, or 20 years after the initial episode. In certain embodiments, one or more peptides and/or peptide salts of the present invention are administered for a period of at least 1 month, or any time period longer, such as at least 6 weeks, 3 months, 6 months, 9 months, 1 year, 2 years, 3 years, or 5 years.

In one embodiment, the present invention provides long-term treatment of chronic pain in patients with non-terminal diseases. In certain embodiments, the present invention provides long-term treatment of chronic pain associated with non-terminal diseases or conditions including, but not limited to, arthritis, neuropathy, low back pain, osteoarthritis, fibromyalgia, headache, and diabetic neuropathy. In one embodiment, the patients treated in accordance with the present invention suffer from chronic pain associated with neurological inflammation and/or neurogenic inflammation. In one embodiment, the patients treated in accordance with the present invention suffer from chronic, noncancer-related pain. As used herein, patients with non-terminal diseases or conditions have a life expectancy of at least 1 year, or any time period longer than 1 year, such as at least 2, 3, 5, 6, 7, 10, 12, 15, 20, or 30 years.

In another embodiment, the peptides of the subject invention are used to treat depression and/or to promote a sense of well being.

In another embodiment, the present invention provides treatment of chronic pain associated with cancer. In another embodiment, the present invention provides treatment of chronic pain associated with HIV/AIDS.

In addition, the present invention provides treatment of acute as well as intermittent pain. In one embodiment, the present invention provides treatment of neurogenic pain. In one embodiment, the present invention can be used to treat patients with break-through pain.

In one embodiment, the present invention can be used to treat severe pain, such as pain associated with trauma, surgery, serious bodily injury, myocardial infarction, cancer, and labor. In other embodiments, the present invention can be used to treat moderate as well as mild pain.

In certain embodiments, the present invention provides treatment of coronary artery disease; diarrhea; schizophrenia; high blood pressure; convulsions; and seizures. The diarrhea may be caused by a number of sources, such as infectious disease, cholera, or an effect or side-effect of various drugs or therapies, including those used for cancer therapy. For applying the peptide salts of the present invention to a human, it is preferable to administer them by parenteral or enteral administration.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression, severity, and/or scope of a condition. In preferred embodiments, the treatment includes reduction or alleviation of pain.

The term “effective amount,” as used herein, refers to an amount that is capable of treating or ameliorating a disease or condition or otherwise capable of producing an intended therapeutic effect. In certain embodiments, the effective amount enables a 5%, 10%, 20%, 30%, or 50% reduction in pain level. The level of pain is commonly rated on a scale of 0 to 10 by the patient.

The peptides and salts of the present invention can also be used to provide anti-inflammatory treatments as described in U.S. 2004/0266805, which is herein incorporated by reference in its entirety.

The peptides and salts of the present invention can also be used to provide treatments for neuroinflammation and/or neurogenic inflammation, as well as related diseases and disorders as described in U.S. patent application Ser. No. 13/113,392, which is herein incorporated by reference in its entirety. Also incorporated herein by reference in its entirety is “How to Design an “Opioid Drug that Causes Reduced Tolerance and Dependence,” 2010, American Neurological Association, Volume 67, No. 5, pages 559-569, authored by Amy Chang Berger and Jennifer L. Whistler.

In one embodiment, the peptides and compositions of the present invention can be used to treat, alleviate, or ameliorate diseases and conditions in which inhibition of substance P (SP) and/or reduction of calcitonin gene-related peptide (CGRP) production is beneficial. In certain embodiments, the peptides and compositions of the present invention can be used to treat, alleviate, or ameliorate diseases and conditions associated with inflammation including, but not limited to, osteoarthritis, asthma, fibromyalgia, eczema, rosacea, migraine, psoriasis, intestinal inflammation, rheumatoid arthritis, neurogenic swelling, edema, bruises, burns, sunburn, meningitis, septic shock, allergy, and dermatitis. In specific embodiments, the peptides and compositions of the present invention can be used to treat, alleviate, or ameliorate diseases and conditions associated with neurological inflammation and/or neurogenic inflammation.

In addition, the peptides and compositions of the present invention can be used to treat, alleviate, or ameliorate inflammation at sites where the primary activating factor is antigen-derived (e.g. bacterial lipopolysaccharide) or of neurogenic origin. In one embodiment, the peptides of the present invention are used to treat pathological inflammatory conditions of the brain.

In addition, the peptides and compositions of the present invention can be used to treat, alleviate, or ameliorate a variety of inflammatory skin conditions, in particular, skin conditions associated with inflammation and pain. The present invention can be used to treat, alleviate, or ameliorate inflammatory skin conditions including, but not limited to, radiation irritation and burns (including UV and ionizing), chemical burns, rhinitis, thermal burns, reddening of the skin, and chemically induced lesions.

The peptides and compositions of the present invention are particularly useful to treat, alleviate, or ameliorate diseases and conditions associated with pain and inflammation including, but not limited to, inflammatory joints, muscles, tendons, nerves and skin; osteo-arthritis and rheumatoid arthritis; dermatitis; inflammatory bowel disease; post-operative pain and inflammation; general blunt trauma; bone injuries; soft tissue infections; and shingles.

### Therapeutic Compositions and Formulations

The present invention further provides therapeutic compositions that contain a therapeutically effective amount of the peptides or salts and a pharmaceutically acceptable carrier or adjuvant. The present invention also contemplates prodrugs or metabolites of the peptides.

As used herein, the terms “pharmaceutically acceptable”, “physiologically tolerable” and grammatical variations thereof, include compositions, carriers, diluents and reagents, are used interchangeably and represent that the materials are capable of administration to or upon a subject such as mammal.

The peptide salts of the present invention may be compounded, for example, with the usual non-toxic, pharmaceutically acceptable carriers for tablets, pellets, capsules, liposomes, suppositories, intranasal sprays, oral solutions, emulsions, suspensions, aerosols, targeted chemical delivery systems (Prokai-Tatrai, K.; Prokai, L; Bodor, N., J. Med. Chem. 39:4775-4782, 1991), and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, liquid or aerosol form, and in addition auxiliary, stabilizing, thickening and coloring agents and perfumes may be used.

Further, the therapeutic composition can comprise one or more peptides or salts of the present invention as a first active ingredient, and one or more additional active ingredients including, but not limited to, co-analgesics such as antidepressant drugs and antiepileptic drugs; and anti-inflammatory compounds known in the art. Such known anti-inflammatory drugs include, but are not limited to, steroidal anti-inflammatory drugs and non-steroidal anti-inflammatory drugs (NSAIDs), including acetylsalicylic acid (aspirin), ibuprofen, acetaminophen, indomethacin, and the like.

In accordance with one embodiment of the invention, therapeutically effective amounts of the peptides of the present invention and the additional active ingredient(s) are administered sequentially or concurrently to a patient. The most effective mode of administration and dosage regimen of the peptides of the present invention and anti-inflammatory agent will depend upon the type of condition to be treated, the severity and course of that condition, previous therapy, the patient's health status, and response to the peptides of the present invention and the judgment of the treating physician. The present compositions may be administered to the patient at one time or over a series of treatments.

The present invention contemplates therapeutic compositions useful for practicing the therapeutic methods described herein. Therapeutic compositions of the present invention contain a physiologically tolerable carrier together with a therapeutically effective amount of a peptide as described herein, dissolved or dispersed therein as an active ingredient.

The peptides used in these therapies can also be in a variety of forms. These include, for example, solid, semi-solid and liquid dosage forms, such as tablets, pills, powders, liquid solutions or suspensions, suppositories, injectable and infusible solutions. The preferred form depends on the intended mode of administration and therapeutic application.

The compositions also preferably include conventional pharmaceutically acceptable carriers and adjuvants which are known to those of skill in the art.

The present peptides and compositions can be in a form that can be combined with a pharmaceutically acceptable carrier. In this context, the compound may be, for example, isolated or substantially pure. The term “carrier,” as used herein, refers to a diluent, adjuvant, excipient, or vehicle with which the compound is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers, particularly for injectable solutions. Particularly preferred pharmaceutical carriers for treatment of or amelioration of inflammation in the central nervous system are carriers that can penetrate the blood/brain barrier. As used herein carriers do not include the natural plants as they exist in nature.

The amount of active ingredient that may be combined with the carrier materials to produce a single dosage form will vary, depending on the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

The peptides of the present invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the present invention. In general, the compositions of the present invention will be formulated such that an effective amount of the bioactive compound(s) is combined with a suitable carrier in order to facilitate effective administration of the composition.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use also can be prepared. The preparation also can be emulsified.

The active ingredient can be mixed with excipients which are pharmaceutically acceptable and compatible with the active ingredient and in amounts suitable for use in the therapeutic methods described herein. Suitable excipients are, for example, water, saline, dimethylsulphoxyde (DMSO) cyclodextrins, dextrose, glycerol, ethanol, sucrose, glucose, mannitol, sorbitol or the like and combinations thereof. In addition, if desired, the composition can contain minor amounts of auxiliary substances such as wetting or emulsifying agents, pH buffering agents and the like which enhance the effectiveness of the active ingredient. Particularly preferred excipients for peptides and compositions of the present invention include dimethylsulphoxyde (DMSO), and hydroxypropyl-β-cyclodextrin.

Liquid compositions also can contain liquid phases in addition to and to the exclusion of water. Exemplary of such additional liquid phases are glycerin, vegetable oils such as cottonseed oil, and water-oil emulsions.

The invention also provides a pharmaceutical pack or kit comprising one or more containers filled with one or more of the ingredients, e.g., compound, carrier suitable for administration.

### Routes of Administration

The peptides and compositions of the present invention can be administered to the subject being treated by standard routes, including the oral, nasal, topical, transdermal, intra-articular, parenteral (e.g., intravenous, intraperitoneal, intradermal, subcutaneous or intramuscular), intracranial, intracerebral, intraspinal, intravaginal, intrauterine, or rectal route. Depending on the condition being treated, one route may be preferred over others, which can be determined by those skilled in the art. In preferred embodiments, the peptides and compositions of the present invention are formulated for oral or parental administration. In another embodiment, the peptides and compositions of the present invention are formulated as a sustained-release formulation.

For instance, the peptides and compositions of the present invention can be topically administered to the subject for treatment of conditions associated with skin inflammation. Compositions for topical administration can be in any of a variety of forms, including suspension, dispersion, solution, ointment, gel, cream, spray, foam, powder, lotion, soak, transdermal patch, solid, micro-particle, vapor, or tape.

The peptides of the present invention may also be administered utilizing liposome technology, slow release capsules, implantable pumps, and biodegradable containers. These delivery methods can, advantageously, provide a uniform dosage over an extended period of time. The amount of the therapeutic composition of the invention which is effective in the treatment of a particular disease, condition or disorder will depend on the nature of the disease, condition or disorder and can be determined by standard clinical techniques.

The dosage of effective amount of the peptides varies from and also depends upon the age and condition of each individual patient to be treated. In general, suitable unit dosages may be between about 0.01 to about 200 mg, about 0.01 to about 100 mg, about 0.01 to about 50 mg, about 0.01 to about 20 mg, about 0.01 to about 10 mg, about 0.01 to about 5 mg, about 0.01 to about 2 mg, or about 0.01 to about 0.2 mg. The dosing regimen for each patient will need to be adjusted individually, taking into account the patient's prior analgesic treatment experience.

In addition, in vitro assays may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

Following is an example that illustrates embodiments for practicing the invention. The example should not be construed as limiting.

### Example 1—Activity of CYT-1010 on Mu-Opioid Receptor Activation

The mu-opioid receptor belongs to the G-protein coupled receptor (GPCR) family. Upon stimulation, beta-arrestin is recruited to the mu-opioid receptor. Activation of the mu-opioid receptor also leads to the inhibition of adenylyl cyclase and a decrease in the concentration of cAMP, which activates a cascade of signalling events that cause both presynaptic inhibition of neurotransmitter release and postsynaptic inhibition of membrane depolarization.

This Example investigates the activity of CYT-1010 on mu-opioid receptor activation. The mu-opioid agonist activity is evaluated based on the effect on beta-arrestin recruitment and the inhibition of cyclic adenosine monophosphate (cAMP) production. The agonist activity of CYT-1010 is compared with that of other commonly-used strong opioid analgesics, such as morphine and met-enkephalin. Morphine is commonly used as the standard of comparison for opioid analgesic drugs.

As shown in the efficacy data and the dose-response profile below, CYT-1010 exerts a maximal analgesic effect, and is significantly more potent and rapid-acting than morphine and met-enkephalin. The data also indicate that the administration of CYT-1010 is associated with lower tolerance, fewer side effects, and less severe physical dependence than other opioid analgesics such as morphine and met-enkephalin.

### Agonist Dose-Response Profile on Beta-Arrestin Recruitment

The effect of mu-opioid receptor agonists on beta-arrestin recruitment is determined using the PathHunter™ beta-arrestin assay. Briefly, PathHunter™ cell lines expressing human mu-opioid receptor (hOPRM₁) are seeded in 384-well microplates at a density of 5,000 cells/well in a total volume of 20 μl, and are allowed to adhere and recover overnight. To each well, 0.2 μl of the mu-opioid receptor agonist selected from CYT-1010, morphine, or met-enkephalin is added at a series of concentrations. The cells are incubated with the mu-opioid receptor agonist at 37° C. for 90 minutes. The experiment is performed in duplicate.

Assay signal is generated through a single addition of 12.5 or 15 μL (50% v/v) of PathHunter™ Detection reagent cocktail, followed by one hour incubation at room temperature. Microplates are read following signal generation with a PerkinElmer Envision™ instrument for chemiluminescent signal detection.

Data are normalized to the maximal and minimal response observed in the presence of control ligand and vehicle. Percentage activity is calculated using the following formula: % Activity=100%×(mean RLU (relative luminescence unit) of test sample−mean RLU of vehicle control)/(mean MAX RLU control ligand−mean RLU of vehicle control)).

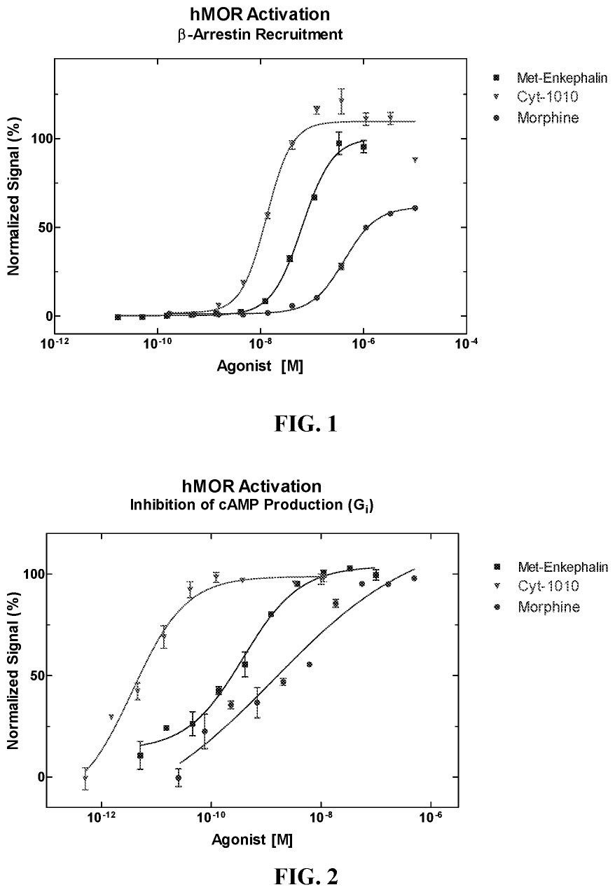

The efficacy data and the dose-response profile of mu-opioid receptor agonists CYT-1010, morphine, and met-enkephalin on beta-arrestin recruitment are shown in FIG. 1 and Table 3.

TABLE 3
Activity of mu-Opioid Receptor Agonists
on Beta-Arrestin Recruitment

| mu-Opioid | EC₅₀ | Max Percent |
|---|---|---|
| Receptor Agonist | (nM) | Activity |
| CYT-1010 | 13.1 | 121.2% |
| Morphine | 422.7 | 60.3% |
| Met-Enkephalin | 64.1 | 100.1% |

Agonist Dose-Response Profile on Inhibition of cAMP Production

The effect of mu-opioid receptor agonists on the inhibition of cAMP production is determined using the PathHunter™ beta-arrestin assay. Briefly, cAMP Hunter™ cell lines expressing human mu-opioid receptor (hOPRM₁) are seeded in 384-well microplates at a density of 10,000 cells/well in a total volume of 20 and are allowed to adhere and recover overnight. To each well, 0.2 μl of the mu-opioid receptor agonist selected from CYT-1010, morphine, or met-enkephalin is added at a series of concentrations. The cells are incubated with the mu-opioid receptor agonist in the presence of 20 μM forskolin at 37° C. for 30 minutes. The experiment is performed in duplicate.

Assay signal is generated through incubation with 20 μL cAMP XS+ ED/CL lysis cocktail for one hour followed by incubation with 20 μL cAMP XS+ EA reagent for three hours at room temperature. Microplates are read following signal generation with a PerkinElmer Envision™ instrument for chemiluminescent signal detection.

Data are normalized to the maximal and minimal response observed in the presence of control ligand and vehicle. Percentage activity is calculated using the following formula: % Activity=100%×(1−(mean RLU of test sample−mean RLU of MAX control)/(mean RLU of vehicle control−mean RLU of MAXcontrol).

The efficacy data and the dose-response profile of mu-opioid receptor agonists CYT-1010, morphine, and met-enkephalin on the inhibition of cAMP production are shown in FIG. 2 and Table 4.

TABLE 4
Activity of mu-Opioid Receptor Agonists
on the Inhibition of cAMP production

| mu-Opioid | EC₅₀ | Max Percent |
|---|---|---|
| Receptor Agonist | (nM) | Activity |
| CYT-1010 | 0.0053 | 98.350 |
| Morphine | 2.7353 | 97.950 |
| Met-enkephalin | 0.2524 | 102.800 |

All patents, patent applications, provisional applications, and publications referred to or cited herein are incorporated by reference in their entirety, including all figures and tables, to the extent they are not inconsistent with the explicit teachings of this specification.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the examples and embodiments described herein are for illustrative purposes only and that various modifications or changes in light thereof will be suggested to persons skilled in the art and are to be included within the spirit and purview of this application.

We claim:

1. A method for treating pain, inflammation, schizophrenia, coronary artery disease, seizure, and/or diarrhea, comprising administering, to a subject in need of such treatment, an effective amount of an isolated peptide or salt thereof, wherein the peptide has a general formula: Tyr-X₁-X₂-X₃,
wherein X₁ is Pro, D-Lys or D-Orn,
X₂ is Trp, Phe or N-alkyl-Phe, wherein alkyl contains 1 to about 6 carbon atoms, and
X₃ is Phe, Phe-NH₂, D-Phe, D-Phe-NH₂ or p-Y-Phe, wherein Y is NO₂, F, Cl or Br;
wherein the subject is a child, is prone to opioid addiction, and/or has chemotherapy-induced pain.

2. The method, according to claim 1, further comprising prior to administration of the isolated peptide or salt thereof, a step of determining whether a subject is a child, is prone to opioid addiction, and/or has chemotherapy-induced pain, wherein the peptide or salt is administered if the subject is a child, is prone to opioid addiction, and/or has chemotherapy-induced pain.

3. The method, according to claim 1, wherein the subject is a human.

4. The method, according to claim 1, wherein the peptide is selected from SEQ ID NOs: 1-26.

5. The method, according to claim 4, wherein the peptide is SEQ ID NO: 13.

6. The method, according to claim 1, wherein the salt is selected from the group consisting of acetate salt, aspartate (L) salt, citrate salt, fumarate salt, hippurate salt, hydrochloride salt, lactate salt, malate (L) salt, mucate salt, phosphate salt, sulfate salt, hemi-sulfate salt, tartrate (L) salt, gluconate (D) salt, maleate salt, and succinate salt.

7. The method, according to claim 1, wherein the salt is selected from the group consisting of maleate salt, hydrochloride salt, lactate salt, aspartate salt, acetate salt, and trifluoro acetate salt.

8. The method, according to claim 1, wherein the peptide or salt thereof is administered for a period of at least 6 weeks.

9. The method, according to claim 1, wherein the subject does not have terminal diseases.

10. The method, according to claim 1, wherein the subject is a child.

11. The method, according to claim 1, wherein the subject is prone to opioid addiction.

12. The method, according to claim 11, wherein the subject has a personal or family history of alcoholism or addiction and/or abuse of psychoactive drugs, suffers from psychiatric conditions, and/or suffers from cognitive impairment.

13. The method, according to claim 1, wherein the subject has chemotherapy-induced pain.

14. The method, according to claim 1, used to treat acute pain.

15. The method, according to claim 1, used to treat neurogenic pain.

16. The method, according to claim 1, used to reduce chronic noncancer pain selected from pain associated with arthritis, neuropathy, low back pain, osteoarthritis, fibromyalgia, headache, and diabetic neuropathy.

17. The method, according to claim 1, used to reduce severe pain associated with trauma, surgery, serious bodily injury, myocardial infarction, and/or cancer.

18. The method, according to claim 1, used to reduce inflammation.

19. The method, according to claim 17, used to reduce neurogenic inflammation.
